(19) United States
(12) Patent Application Publication
(10) Pub. No.: US 2008/0274150 A1
(43) Pub. Date: Nov. 6, 2008

(54) Process For Removing Odors From Hydrocarbons

(76) Inventors: Mark Dierker, Dusseldorf (DE); Jurgen Falkowski, Monheim (DE)

Correspondence Address: FOX ROTHSCHILD LLP, 1101 MARKET STREET, PHILADELPHIA, PA, 19107, US

(21) Appl. No.: 12/093,711

(22) PCT Filed: Nov. 15, 2006
(86) PCT No.: PCT/EP06/10946

§ 371 Date: May 14, 2008

(30) Foreign Application Priority Data
Nov. 16, 2005 (EP) 05024998

(51) Int. Cl. A61K 8/18

(52) U.S. Cl. 424/401; 585/853

(57) ABSTRACT

A process for removing odors from hydrocarbons is disclosed. Odor-free hydrocarbons obtained by the process are described, which hydrocarbons are particularly suitable for use in cosmetic applications. Cosmetic formulations which contain the odor-free hydrocarbons are also described.

### CROSS-REFERENCE TO RELATED APPLICATIONS

This application claims priority under 35 U.S.C. Section 119 of EP 05024998 filed Nov. 16, 2005 and International Application PCT/EP2006/010946 fled Nov. 15, 2006, the entire contents of which are incorporated herein by reference in their entireties.

### FIELD OF THE INVENTION

This invention relates to a process for removing unwanted odors from liquid hydrocarbons and to the use of the odor-free hydrocarbons in cosmetic compositions.

### BACKGROUND OF THE INVENTION

Readily-volatile oil components, also known as light emollients, are used in a number of formulations by the cosmetics industry. Large quantities of readily volatile components are used in particular for make-up and in personal care formulations. These components may be, for example, volatile cyclic silicones (for example, cyclopentasiloxane or cyclomethicone) or hydrocarbons from petrochemical processes. The hydrocarbons, because of their production, are predominantly mixtures of linear and branched hydrocarbons Examples and application-related descriptions of such formulations can be found in standard works, such as, for example: “Handbook of Cosmetic Science and Technology”, A Barel, M. Paye, H. Maibach, Marcel Dekker Inc., 2001. All the raw materials described have to meet the high quality requirements in cosmetic formulations. Besides having to be toxicologically safe, these raw materials must not contain any residues of quality-reducing components which would lead, for example, to odor impairment of the cosmetic formulation.

The problem addressed by the present invention was to provide light emollients which, on the one hand, would be toxicologically safe and which, on the other hand, could be used without any restrictions in typical cosmetic formulations. In many cases, simple linear, saturated hydrocarbons, which can be obtained, for example, by hydrogenation of olefins, meet this requirement profile. However, it has been found that, after a distillation step to obtain the required properties in regard to purity and volatility, these raw materials have an unacceptable smell for use in cosmetic formulations.

High-purity linear hydrocarbons, which are liquid at room temperature and which have been purified by very complicated laboratory processes, such as chromatographic separation processes for example, are substantially odorless. The remaining smell of such hydrocarbons, however, cannot be completely eliminated by a deodorizing step carried out in known manner with inert gases, such as steam or nitrogen. The smell is probably caused by unquantifiable impurities formed during the complex production process. The production processes for unbranched higher olefins are mostly oligomerizations of lower hydrocarbons, such as for example the oligomerzation of ethene in synthesis reactions to form so-called Ziegler olefins, or processes using organometallic mixed catalysts, such as Shell's SHOP process. Branched higher olefins are preferably produced by oligomerization or co-oligomerization of lower olefins, such as propene, isobutene and n-butene, using mainly acidic catalysts, as for example in the Bayer process for isobutene, or using organometallic catalysts.

It has now surprisingly been found that hydrocarbons can be freed from troublesome odors by the process of the present invention.

### BRIEF SUMMARY OF THE INVENTION

Accordingly, the present invention provides a process for removing odors from liquid hydrocarbons, which process comprises:
(a) contacting an oil phase containing hydrocarbons having an odor with an aqueous alkaline phase;
(b) mixing the oil phase with the aqueous alkaline phase; wherein when the process is a discontinuous process, power input into (b) is at least 2 W/kg and when the process is a continuous process, energy input into
(b) is at least 1 kJ/kg; and
(c) separating the oil phase from the aqueous phase; said oil phase containing hydrocarbons which are odor-free.

Another embodiment of the present invention then is hydrocarbons which are odor-free and particularly suitable for use in cosmetic compositions. The hydrocarbons obtained by way of the present invention are also free of deodorizing compounds.

Another aspect then of the present invention is cosmetic compositions can be provided which contain the odor-free hydrocarbons obtained by way of the present invention, which hydrocarbons are especially suitable for use in cosmetic applications.

The term “odor-free” as used herein shall be understood to mean that the hydrocarbons obtained by way of the present invention are odor-free to the extent that they are suitable for use in cosmetic compositions.

### DETAILED DESCRIPTION OF THE INVENTION

The process according to the invention is suitable not only for liquid hydrocarbons, but also and preferably for hydrocarbons which are liquid at room temperature, i.e. 21° C. However, the process can also be carded out at higher temperatures, for example, when relatively high-melting hydrocarbons are to be purified.

Hydrocarbons are understood in the following to be alkanes and alkenes as both linear and branched isomers, but also ring-closed hydrocarbons and mixtures thereof with one another.

It is already known that hydrocarbons can be contacted with lyes and thus purified. U.S. Pat. No. 1,553,141 (Clark) describes a process in which impurities may be removed from natural or synthetic oils by mixing with an aqueous alkaline solution. However, the patent does not refer to the possibility of improving the odor of the oils, nor does it provide any guidance on the energy input that would be necessary to accomplish that. In addition, U.S. Pat. No. 1,553,141 is not concerned with hydrocarbon mixtures for use in cosmetic compositions.

U.S. Pat. No. 1,961,324 (Bosing) describes a process for removing odors from hydrocarbons in which the hydrocarbons are contacted with an aqueous lead oxide solution and the lead is precipitated as a sulphide by the subsequent addition of sulfur.

The process according to the invention provides that the hydrocarbon or oil phase is sufficiently mixed with the aqueous alkali phase, i.e., that an adequate phase interface is established between these two phases which are insoluble in one another. To establish the phase interface, a sufficient amount of energy, preferably mechanical energy, is introduced into the liquid/liquid system.

Discontinuous processes are characterized in that the energy is introduced into the reaction mixture as a whole. Special dispersing stirrers can normally be used for this purpose in discontinuous processes carried out in stirred tank reactors. The power required in [W] for a stirred system can be calculated, for example, using the following formula:

P=Ne×ρ×n³×d⁵

where N is the Newton value dependent on the geometry of the stirrer and the Reynold's number, ρ is the mean density in [kg/m³] of the stirred system, n is the stirrer speed in [l/s], and d is the stirrer diameter in [m].

In order to be able to compare the energy input for continuous and discontinuous processes using various dispersion systems, a specific energy input Q in (J/kg) is defined which describes the energy input per unit of weight.

Discontinuous processes according to the invention may utilize a specific power input of at least 2, preferably at least 5, and more preferably at least 10 W/kg. A specific power input of 2 to 200 W/kg is preferred, while a specific power input of 5 to 100 W/kg is particularly preferred. For a discontinuous process, this specific energy input Ps can be calculated by energy input per stirring time as follows:

Ps[W/kg]=specific energy input Q[J/kg]/stirring time [s].

Accordingly, the energy input for a discontinuously-stirred system, as calculated on the basis of the above formula, is:

Q=(P×t)/m

where t is the stirring time in [s] and m is the weight of the stirred system in [kg]. In order to obtain the specific power input according to the invention for discontinuous processes, the energy input and the stirring times may be varied. Typical energy inputs may be selected, for example, between 1 and 100 kJ/kg. One of skill may then select the times in which the two phases may be contacted with one another as a function of the specific power input required. These times may vary accordingly, preferably between 1 and 300 minutes, and more preferably between 1 and 60 minutes or between 1 and 30 minutes. Conversely, with predetermined stirring times, one of skill can select the energy input required to obtain the specific power input required.

With conventional stirring processes (i.e., stirring processes which do not correspond to the invention), the specific power input for low-viscosity systems is typically 0.1 to 1 W/kg.

Continuous processes are characterized in that the energy is introduced continuously into part of the reaction mixture as a whole. Toothed-rim dispersion machines, colloid mills, and high-pressure homogenizers may generally be used for continuous processes. For a continuous system, the energy input Q may be calculated analogously to:

Q=P/ms

where ms in the mass flow of the two-phase system is in [kg/s]. If the process according to the invention is carried out as a continuous process, an energy input of at least 1 kJ/kg, preferably at least 2 kJ/kg and more particularly at least 5 kJ/kg is especially suitable for the two-phase system. The energy input is preferably between 1 and 100, more preferably between 2 and 70, most preferably between 5 and 60 kJ/kg and, in a most particularly preferred embodiment, between 5 and 45 kJ/kg.

By contrast, conventional continuous processes which do not correspond to the invention have a far lower energy input, with values below 1 kJ/kg being typically utilized.

Although a considerably greater specific power input (discontinuous processes) or a considerably greater energy input (continuous processes) under the temperature and concentration limits explained above is possible because the hydrocarbon to be purified is substantially chemically inert under these process conditions, it does lead to uneconomically high energy and equipment costs. Accordingly, it is preferred to limit the maximum specific power input in discontinuous processes to 200 W/kg and the maximum energy input in continuous processes to 100 kJ/kg.

The alkali treatment is followed by oil and water phase separation and—irrespective of whether the process was carded out as a discontinuous process or as a continuous process—the hydrocarbon phase may then be freed from the remaining quantity of alkali solutions, for example, by addition of deionized water and subsequent phase separation. In a preferred embodiment of the process according to the invention, the hydrocarbon phase is purified after removal of the aqueous phase by distillation.

The process according to the invention is preferably carried out using dilute lyes. In principle, any lyes containing at least one cation from the group of alkali and alkaline earth metals may be used, with soda lye or potash lye preferably being used. Lead oxide or other water-soluble lead compounds are not utilized in the process.

The lyes may be used in a concentration range from 0.1% to the solubility limit of the corresponding alkali metal or alkaline earth metal hydroxide in water. The preferred concentration range is between 2 and 60% by weight, preferably between 3 and 50% by weight, and more preferably between 5 and 20% by weight. Solutions of metal hydrides, such as sodium borohydride or lithium aluminium hydride, for example, may be used instead of simple alkali solutions. More particularly, an industrially suitable solution of 12% by weight lithium aluminium hydride and 40% by weight sodium hydroxide in 48% by weight deionized water, which is known commercially as Venpure™Solution borohydride reducing agent, may be used in the above-described concentrations for the process according to the invention.

In principle, the treatment with the dilute alkali solution may be carried out at a temperature in the range of from 0 to 250° C., preferably at a temperature in the range of from 15 to 150° C., and more preferably at a temperature in the range from 20 to 100° C., this temperature range advantageously being used, in particular, in an industrial reactor. The most particularly preferred temperature range is between 40 and 80° C.

The hydrocarbons may be unsaturated or, preferably, saturated hydrocarbons which have been produced by hydrogenation from the corresponding unsaturated compounds. The hydrocarbons may be linear, branched or cyclic in structure and may also be physical mixtures of linear, branched or cyclic hydrocarbons. In the molecular structure of the saturated or unsaturated hydrocarbons, linear, branched and cyclic structures may also be present together in any combination, i.e. for example a saturated hydrocarbon ring with an unsaturated linear substituent. Hydrocarbon compounds containing 6 to 30 carbon atoms, and preferably 8 to 20 carbon atoms, may be treated by the process according to the invention. Hydrocarbons liquid at room temperature are particularly preferred for the process according to the invention.

In another preferred embodiment, the quantity ratio of the hydrocarbon oil phase and the alkaline aqueous phase is in the range from 100:1 to 10:1 (m/m).

The invention has the following advantages:
Hydrocarbons may be used as an inexpensive raw material source for the production of light emollients for cosmetic applications.
The treatment process according to the invention can be carried out with little technical difficulty.
Product losses are lower by comparison with the deodorization of readily volatile products.
The long-term stability of the products in terms of odor is distinctly better in comparison with deodorized products because the unknown or unquantifiable odor sources may be effectively removed by way of the invention.

The present invention also relates to a hydrocarbon mixture, liquid at 21° C., containing saturated or unsaturated, unbranched or cyclic hydrocarbons which mixture has been treated by the process described above, the hydrocarbon being free from deodorizing compounds such as, for example, zinc ricinoleate, zinc stearate, aluminium hydroxychloride, essential oils, and perfumes.

The odor-free hydrocarbons according to the invention may advantageously be used in cosmetic compositions, and are preferably used for the production of stable cosmetic emulsions. The cosmetic compositions may be body care formulations, for example in the form of creams, milks, lotions, sprayable emulsions, products for eliminating body odor, etc. The hydrocarbons purified in accordance with the invention may also be used in surfactant-containing formulations such as, for example, foam and shower baths, hair shampoos and care rinses. The cosmetic compositions may be present in the form of emulsions or dispersions which contain the water and oil phases alongside one another. Preferred cosmetic compositions are those in the form of a w/o or o/w emulsion with the usual concentrations—familiar to one of skill in the art—of oils/fats, waxes, emulsifiers, water and the other auxiliaries and additives typically utilized in cosmetic products.

A cosmetic composition typically contains 1 to 50% by weight, preferably 5 to 40% by weight and more preferably 5 to 25% by weight of oil components which—together with, for example, oil-soluble surfactants/emulsifiers and oil-soluble components—are part of the so-called oil or fatty phase. The oil components include fats, waxes and liquid oils, such as hydrocarbons for example, but not emulsifiers/surfactants. The hydrocarbons obtained by way of the present invention may be present as the sole oil component or in combination with other oils/fats/waxes. The percentage content of at least one hydrocarbon, based on the total quantity of oil components, is preferably 0.1 to 100% by weight and, more particularly, 1 to 50% by weight. Quantities of 1 to 20% by weight are preferred, while quantities of 3 to 20% by weight are particularly preferred.

Depending on the application envisaged, the cosmetic formulations contain a number of other auxiliaries and additives such as, for example, surface-active substances (surfactants, emulsifiers), other oil components, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, anti-dandruff agents, film formers, swelling agents, insect repellents, self-tanning agents, tyrosinase inhibitors (depigmenting agents), hydrotropes, solubilizers, preservatives, perfume oils, dyes, etc. The quantities in which these additives are used may be determined by the intended use. Typical cosmetic compositions contain between 0.1 and 20% by weight, preferably between 1 and 15% by weight, and more preferably between 1 and 10% by weight of a surface-active substance or a mixture of surface-active substances.

The following examples are illustrative of the present invention and should not be construed in any manner whatsoever as limiting of the scope thereof.

### EXAMPLES

### 1. High-Energy-Input Purification of Dodecane—Discontinuous Process

1 kg of the reaction product dodecane with an iodine value (IV) of 0.04, which had been produced by hydrogenation from dodecene, was introduced into a laboratory stirred tank reactor after filtration of the hydrogenation catalyst and heated to 60° C. 0.5 kg of a 10% soda lye was then added, and the entire two-phase system was heated with stirring to 60° C. Using an Ultra-Turrax (type: IKA T50; max. power: 1.1 kW at 10,000 r.p.m.), which was fitted with a dispersing disk as stirrer, the whole was then stirred for 5 mins. at 6,000 r.p.m. This corresponds to a specific power input of about 160 W/kg and an energy input of about 47 kJ/kg. After phase separation, the upper phase was washed with 0.5 kg deionized water and dried in vacuo. The hydrocarbon thus produced can be used unconditionally in terms of troublesome odors for cosmetic formulations.

### 2. Low-Energy-Input Purification of Dodecane—Discontinuous Process

Using a conventional laboratory stirrer (model: IKA RW 20 DZM; max. power 70 W at 500 r.p.m.), the same amounts of hydrocarbon and alkali, as in Example 1, were stirred for 30 mins. at 100 r.p.m. at a temperature of 60° C. This corresponds to a specific power input of 0.4 W/kg and an energy input of about 0.7 kJ/kg.

A similar energy input would also be achieved in an industrial reactor using, for example, 10,000 kg hydrocarbon, providing this system was also stirred for 30 mins. with a stirrer delivering 5 kW. After phase separation, the upper phase was washed with 0.5 kg deionized water and dried in vacuo. The hydrocarbon thus produced maintained an odor and was thus unsuitable for cosmetic formulations.

### 3. Purification of Dodecane by Deodorization

1 kg of the hydrocarbon used in Example 1 was introduced into a laboratory stirred tank reactor equipped with a distributor for inert gas and heated to 80° C. A 1 Nm³/h stream of nitrogen was then passed through the hydrocarbon for 1 hour under a vacuum of 100 mbar for deodorization. The vacuum was then broken, the nitrogen was turned off, and the product was cooled. The hydrocarbon thus produced still maintained an odor and was thus unsuitable for cosmetic formulations.

In summary, Example 1, representative of the present invention, demonstrated that hydrocarbons were obtained which were odor-free and suitable for use in cosmetic formulations in comparison with Examples 2 and 3, which used low-energy input and deodorization, respectively, with the resultant hydrocarbons maintaining an odor, which odor rendered the hydrocarbons unsuitable for use in cosmetic formulations.

What we claim is:

1-17. (canceled)

18) A process for removing odors from hydrocarbons, which process comprises:
(a) contacting an oil phase containing hydrocarbons having an odor with an aqueous alkaline phase;
(b) mixing the oil phase with the aqueous alkaline phase; wherein when the process is a discontinuous process, power input into (b) is at least 2 W/kg and when the process is a continuous process, energy input into (b) is at least 1 kJ/kg; and
(c) separating the oil phase from the aqueous phase; said oil phase containing hydrocarbons which are odor-free.

19) The process of claim 18 wherein the process is a discontinuous process.

20) The process of claim 19 wherein the power input into (b) ranges from 2 to 200 W/kg.

21) The process of claim 18 wherein the process is a continuous process.

22) The process of claim 21 wherein the energy input into (b) ranges from 1 to 100 k J/kg.

23) The process of claim 18 wherein the process is carried out at a temperature of from 20 to 100° C.

24) The process of claim 18 wherein the aqueous alkaline phase contains alkali and/or alkaline earth metals.

25) The process of claim 18 wherein the aqueous alkaline phase contains a metal hydride.

26) The process of claim 25 wherein the metal hydride is sodium borohydride or lithium aluminum hydride.

27) The process of claim 18 wherein the aqueous alkaline phase contains from 2 to 60% alkaline compounds by weight.

28) The process of claim 27 wherein the aqueous alkaline phase contains from 5 to 20% alkaline compounds by weight.

29) The process of claim 18 wherein the hydrocarbons are saturated or unsaturated linear or branched or cyclic hydrocarbons of from 6 to 30 carbon atoms, and mixtures of thereof.

30) The process of claim 29 wherein the hydrocarbons are of from 12 to 18 carbon atoms.

31) The process of claim 18 wherein the hydrocarbons and aqueous alkaline phase are present in a ratio of from 100:1 to 10:1 (m/m).

32) The process of claim 18 wherein the mixing is carried out for a time of from 1 to 300 minutes.

33) The process of claim 32 wherein the mixing is carried out for a time of from 1 to 30 minutes.

34) The process of claim 18 which further comprises distilling the odor-free hydrocarbons.

35) The process of claim 18 wherein the odor-free hydrocarbons are free of deodorizing compounds.

36) Hydrocarbons obtained by the process of claim 18, which hydrocarbons are odor-free and free of deodorizing compounds.

37) A cosmetic composition which comprises an emulsion containing odor-free hydrocarbons which are free of deodorizing compounds.
